(19) United States
(12) Patent Application Publication
(10) Pub. No.: US 2023/0298713 A1
(43) Pub. Date: Sep. 21, 2023

(54) TIME VARIABLE CRITICAL PATH SYSTEM AND METHOD OF PATIENT CARE

(71) Applicants: Memory Kashumba, Tampa, FL (US); Ano Kashumba, Tampa, FL (US)

(72) Inventors: Memory Kashumba, Tampa, FL (US); Ano Kashumba, Tampa, FL (US)

(21) Appl. No.: 18/055,056

(22) Filed: Nov. 14, 2022

Related U.S. Application Data
(63) Continuation of application No. 16/421,154, filed on May 23, 2019, now abandoned.
(60) Provisional application No. 62/676,038, filed on May 24, 2018.

(51) Int. Cl. G16H 10/60; G16H 50/30; G16H 80/00; G16H 15/00

(52) U.S. Cl. CPC G16H 10/60; G16H 50/30; G16H 80/00; G16H 15/00; A61B 5/7275

(57) ABSTRACT

A system and a method of using time and time intervals to coordinate care activities and generate automated triggered instructions.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention and the applicants make no claim to any trademarks referenced.

### CROSS-REFERENCE TO RELATED APPLICATION

This application claims priority from pending U.S. patent application Ser. No. 16/421,154, filed May 23, 2019, which claims priority from U.S. Provisional Patent Application No. 62/676,038, filed May 24, 2018, both of which are titled TIME VARIABLE CRITICAL PATH SYSTEM AND METHOD OF PATIENT CARE and both of which are hereby incorporated by reference in its entirety herein for all purposes.

### BACKGROUND OF THE INVENTION

### Field of the Invention

The invention relates to medical information system, and, more particularly to information as to patient procedure status.

### Description of Related Art

Current medical care is provided to a patient by multiple medical providers without a common source. This leads to delay in patient care and lack of patient satisfaction. Respective data is used to attempt to alleviate the delays as well as discover factors contributing to patient satisfaction. Large amount of time is spent with telephone communication, multiple logins to check for test results, providers seeing multiple patients and then communicating plan of care after their rounds.

### BRIEF SUMMARY OF THE INVENTION

The invention in one form is directed to a system and associated method for autonomously managing patient care across multiple disciplines using a common source of communication that is autonomously generated.

This invention relates to a specific scientific system and method for automated communication and delivery of patient care across multiple disciplines in a hospital ward. The modern hospital environment has multiple caregivers attending to one patient and all prescribing treatment and testing which is specific to the protocols within the caregivers' specialty. The patient, hospital staff and consulting practitioners specifically need a communication method which provides detail information with respect to what procedures and tests have been ordered and the initiation time of the order versus the age of the order. More specifically one needs to create a communication path between software programs that is based on a treatment plan that follows scientific evidence-based algorithms is needed to ensure that procedures and treatments are delivered to the patient in a timely fashion and limits those treatments and procedures to those clinically necessary for the patient's state.

Therefore, what is needed in the art is a system that collects and disseminates information to hospital and clinical staff about treatment plans, procedures and time of order versus time of completion for various tests, procedures, and therapies prescribed by both hospitalist and consultants such as neurologist and cardiologist.

The invention includes any Electronic Medical Records (EMR) database and data transformational tool that communicates to time variable critical path evidence-based display tool through a pathway that is determined by the application of evidence-based medicine known as the critical path in order to generate times and time intervals.

The invention also includes the generation of visual indicators on the time taken to provide care which may include alerts, alarms, buttons and highlighting colors.

The invention provides the caregiver the critical path and the time taken to give medical care combined into a single visual path which is referred to as the Time variable critical path.

The time variable critical path communicates to a business intelligence tool-to generate times and time intervals depending on the treatments and procedures needing to be delivered to the patient to ensure that the patient is processed efficiently in the hospital and that the patient treatment and stay are coordinated with the payers preauthorized treatment cycle.

The method of assigning the critical path involves studying and applying evidence-based studies and establishing set tasks that must be done to treat a patient with a specific diagnosis. These critical path steps must be performed in order for a patient to be considered ready for discharge.

The time variable critical path display tool is a displayed monitor that can also be transformed to other types of visual displays including hand held devices such as smart phones, tablets, computers, laptops, tv's or any other display device.

The invention in another form provides the hospitalist a total view of the prescribed treatment and procedures for a specific patient and the scheduling of the treatments and procedures with respect to the timing of providing the treatments and procedures as well as tracking them to the anticipated discharge date.

In yet another form of the invention, the information is collected from the primary input systems and EMR system without requiring secondary input thereby minimizing the work load on the caregivers. An advantage of the present invention is to provide the overall patient prescribed therapies in view of the anticipated length of stay.

Another advantage of the present invention allows the hospitalist to review therapies and procedures that have been ordered and the timing of the procedures with respect to anticipated discharge date and to determine based on best practices if the prescribed therapy or procedure is applicable to the patient's condition.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 overall system diagram.

FIG. 2 typical system dashboard display for displaying patient status and procedures and therapies ordered.

FIG. 3 typical system dashboard.

FIG. 4 alternative system dashboard display for displaying patient status and procedures and therapies ordered.

FIG. 5 alternative system dashboard.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, may be arranged, substituted, combined, and designed in a wide variety of different configurations, all of which are explicitly contemplated and made part of this disclosure.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

The following terms as used within the description of the invention are defined:

The use of the term “patient” is an individual that is either admitted, under care, or under observation in a hospital, etc.

The use of the term “Hospital” is a patient care facility.

The use of the term “Patient care facility” and “healthcare facility” is used interchangeably and includes any of the following emergency department, intensive care unit, surgical ward, non-surgical ward, observation ward, outpatient care facility, catheter laboratory, anesthetics, cardiology, diagnostic imaging, ear nose and throat department, elderly services department, gastroenterology, general surgery, day surgery, hematology, hospital laboratories, maternity departments, microbiology department, nephrology, neonatal unit, neurology, nutrition and dietetics, diabetes, cancer, kidney problems, pediatrics, elderly care, surgery and critical care, gastroenterology, obstetrics and gynecology units, occupational therapy, oncology, ophthalmology, orthopedics, pain management, pharmacy, physiotherapy, radiotherapy, renal unit, rheumatology, genitourinary medicine, urology, or any care facility within a hospital, etc.

The use of the term “caregiver” is a hospital employee including hospitalist, doctor, registered nurse, licensed nurse, nurses aid, physician assistant, phlebotomist, x-ray technician, ultrasound technician, MRI operator, CT scan operator, hospital administrator, room service personnel, therapist, physical therapist, speech therapist, billing service personnel, infection control personnel, and any other patient service personnel employed by the hospital, etc.

The use of the term hospitalist refers to a dedicated in-patient physician who works exclusively in a hospital.

The use of the term dashboard can alternatively be called a dashboard display and within the disclosure the terms are used interchangeably.

The invention is used by the caregiver to view all the prescribed therapies and procedures that a patient has been prescribed and determine a critical path for the prescribed therapies and procedures so that the amount of time spent in the hospital is minimized and the patient is discharged at the appropriate time. This is important because the average hospital cost per inpatient day in the United States is approximately $1200-$2500 per day. Therefore, minimizing the time, a patient spends in a hospital provides multiple benefits for the patient, the payer, and the hospital. The invention is primarily used when a patient has a critical illness or injury which is defined as a condition that acutely impairs one or more vital organ systems such that there is a high probability of imminent or life-threatening deterioration in the patient's condition (e.g., including but not limited to, central nervous system failure; circulatory failure; shock; renal, hepatic, metabolic, and/or respiratory failure).

Caregivers and hospitalists providing care to the critically ill patient must perform highly complex decision making and interventions of high intensity that are required to prevent the patient's inevitable decline.

The benefits of shrinking the timeline a patient spends in the hospital can produce the following benefits for the various individuals and organizations. For the patient it can reduce their deductible and pharmacy cost as well as facilitating a transfer from the healthcare facility to a lower cost facility such as the patients home, for the hospital it streamlines the process, so the patient can be discharged within the payors discharge schedule for the patient. This is important because in many cases the hospital is not reimbursed for excess hospital time over the payors schedule. It also frees up resources for use by other patients. For the payor it helps them manage cost and makes the inpatient cost more predictable.

This disclosure is drawn to methods and systems related to displaying and visualizing medical procedure and therapy information for facilitating effective analysis by medical professionals. While an exemplary embodiment may be utilized in an intensive care unit of a medical facility, any aspect of medical care (such as a psychiatric facility, general practice environments and home-based medical care, for example) may utilize the methods and systems described herein. Further, although the methods and systems are generally described herein in relation to medical care this invention would be applicable to any process with time-based deliverables that are needed to be completed within a limited time span.

The invention provides the care givers with a time variable critical path that allows the caregiver to view all the procedures or therapies that have been prescribed for a patient and system that provides valuable assistance for data analysis and decision making for the caregiver or Hospitalist. How the caregiver perceives and interacts with patient data may affect their ability to arrive at the best critical care solution. The medical field has provided excellent opportunities for the application of data visualization technology with the potential to help improve healthcare that can improve health-related outcomes. In a care context, the clinical decision-making process should always be supported by relevant and reliable clinical data. In this case, the use of the instant invention may be considered a data analysis method in which caregivers are able to view all the prescribed therapies and procedures and determine when each one had been prescribed and if they have been done or when they are scheduled. The instant invention also gives the caregiver the ability to review the prescribed therapies and procedures to determine if they are appropriate and clinically relevant and how the prescribed therapies and procedures fit together with respect to the available time to ensure that the patient stay is synchronized with the target discharge date.

When the prescribed therapies and procedures are performed in an optimal sequence the diagnosis time is reduced and patient care improves. The instant invention allows the caregiver to look at the prescribed therapies and procedures and triage the prescribed therapies and procedures using the best practices which are based on improved outcomes and evidence-based factors. This allows the caregiver to better obtain the analysis results and draw conclusions.

The invention in one form is directed to a system and associated method for autonomously managing patient care across multiple disciplines using a common source of communication that is autonomously generated.

The invention includes any Electronic Medical Records (EMR) database and data transformational tool that communicates to a time variable critical path evidence-based display tool through a pathway that is determined by the application of evidence-based medicine known as the critical path in order to generate times and time intervals.

The invention also includes the generation of visual indicators on the time taken to provide care which may include alerts, alarms, buttons, and highlighting colors.

The invention provides the caregiver the critical path and the time taken to give medical care combined into a single visual path which is referred to as the time variable critical path.

The time variable critical path can communicate with a business intelligence tool to generate times and time intervals based on which treatments and procedures are needed to be delivered to the patient to ensure that the patient is processed efficiently in the hospital and that the patient's treatment and stay are coordinated with the payers preauthorized treatment cycle.

The method of assigning the critical path involves studying and applying evidence-based studies and establishing set tasks that must be done to treat a patient with a specific diagnosis. These critical path steps must be performed in order for a patient to be considered ready for discharge.

The time variable critical path display tool is a displayed monitor or dashboard display that can also be transformed to other types of visual display including hand held devices such as smart phones, tablets, computers, laptops, tv's or any other display device.

The invention allows the caregiver to see which procedures associated with each procedure id have been completed, which are pending, and which are delayed. This allows the caregiver to take the appropriate action to ensure that the patient is released within the prescribed times set by the payers, hospital administrators, best clinical practices and caregiver knowledge.

The invention is directed to a system and associated method for autonomously managing patient care across multiple disciplines using a common source of communication that is autonomously generated.

Autonomously generated is defined as:
Times and time interval,
New data based on interpretation of the times and time intervals,
Communication with all care givers regarding patient care.

An example is a patient admitted for chest pain who requires a lab test known as a troponin to be completed in 6 hours. The instant invention would measure the time the order is placed, it identifies if the order is not carried out at 6 hours, at a pre-determined interval after the delay the system communicates with the lab technician that the test is late. The system rechecks in 10 minutes if the test has been completed and notifies the technician again. It rechecks again at a specified time interval and if the test is completed the loop ends, however, if not completed the autonomous system communicates with the lab technician, their supervisor, and the hospitalist. That data is then used for analysis.

The invention is compatible to work with any Electronic Medical Records (EMR) database due to the use of the inventions interface engine and data transformational tool that communicates to the time variable critical path evidence-based display tool through a pathway that is determined by the application of evidence-based medicine known as the critical path in order to generate times and time intervals. The generation of critical pathways through the application of evidenced based medicine combined with the critical pathway of a hospital-based process like the acquisition of the troponin blood test for a chest pain patient.

The invention also includes the generation of visual indicators on the time taken to provide care which may include alerts, alarms, buttons, and highlighting colors.

The invention provides the caregiver the critical path and the time taken to give medical care combined into a single visual path which is referred to as the time variable critical path.

The time variable critical path communicates to a business intelligence tool to generate times and time intervals which the treatments and procedures are needed to be delivered to the patient to ensure that the patient is processed efficiently in the hospital and that the patient treatment and stay are coordinated with the payers preauthorized treatment cycle. The invention will communicate to any user interface based on the customers preference.

The method of assigning the critical path involves studying and applying evidence-based studies and establishing set tasks that must be done to treat a patient with a specific diagnosis. These critical path steps must be performed in order for a patient to be considered ready for discharge.

The time variable critical path display tool is a displayed monitor that can also be transformed to other types of visual display including hand held devices such smart phones, tablets, computers, laptops, tv's or any other display device.

Methods for displaying and visualizing medical information for facilitating effective analysis by medical professionals are known in the art. Procedures or methods for visualizing medical information have been described in the following U.S. patents and patent applications, U.S. Pat. No. 8,170,888, entitled Method and system for assessing, quantifying, coding and communicating a patient's health and perioperative risk, U.S. Pat. No. 8,645,164, entitled Medical information visualization assistant system and method, U.S. Pat. No. 8,930,214, entitled Consolidated healthcare and resource management system, U.S. Patent Application 20170293700, entitled Patient state representation architectures and uses thereof, U.S. Patent Application 20030130872, entitled Methods and apparatus for managing and using inpatient healthcare information The foregoing patents and applications are hereby incorporated herein by reference, in their entireties.

An important differentiator between the instant invention and other visualization methods is that the instant invent populates the data to form the time variable critical path dashboard from the various systems currently employed within the health care facility. All inputs come from the connected systems and it is collated so that it can be displayed in one easy to view critical path chart. To achieve this the invention is connected to any of the systems that are utilized in the care facility including but not limited to the pharmacy, Electronic Medical Records (EMR), neurology, intensive care unit, surgical ward, non-surgical ward, observation ward, outpatient care facility, catheter laboratory, anesthetics, cardiology, diagnostic imaging, ear nose and throat department, elderly services department, gastroenterology, general surgery, day surgery, hematology, hospital laboratories, maternity departments, microbiology department, nephrology, neonatal unit, neurology, nutrition and dietetics, diabetes, cancer, pediatrics, elderly care, surgery and critical care, gastroenterology, obstetrics and gynecology units, occupational therapy, oncology, ophthalmology, orthopedics, pain management, physiotherapy, radiotherapy, renal unit, rheumatology, genitourinary medicine and Urology systems.

The instant invention is a method for monitoring medical procedures wherein a caregiver enters a medical order on a remote computer that is in electronic communication with an electronic medical record server. The electronic medical record server stores the medical order in an electronic medical record database on a storage media electronically in communication with the electronic medical record server and the electronic medical record database is in electronic communication with an interface engine using said electronic medical record server. The interface engine is also in electronic communication with a rules database and the rules database is configured to store rules for using the medical order stored in the electronic medical record database the rules creating dashboard data set comprising of an alert, time, and procedure id.

The interface engine is in electronic communication with a second database server and the second database server is in electronic communication with dashboard data to storage.

The second database server then stores said the dashboard data set on the dashboard database storage;

The second database server is also in electronic communication with a dashboard display and the second database server communicating dashboard data set comprising of alerts, time and the procedure id to a dashboard display;

The second database server being in electronic communication with a smart phone or tablet and communicating the dashboard data set comprising of an alert, a time and the procedure id to said smart phones, tablets, computers, laptops, tv's or any other display device.;

The caregiver can then read dashboard data set on either the smart phone or tablet and the dashboard display.

The electronic communication means can be selected from anyone of the following protocols including but not limited to wireless communications such as Wi/FI, Zigbee, RFID, NFC, and Bluetooth as well any form of hardwired communication.

The invention manifested in the dashboard/dashboard display ensures that all diagnostic procedures have been completed within a fixed period to ensure that the patient receives the various tests, procedures, and therapies prescribed by caregiver, hospitalist, and consultants such as neurologist and cardiologist that need to provide an informed diagnosis and it ensures that the care facility does not have to keep the patient for an extended length of stay where the extended time period is not reimbursed by the payer which include state, federal, and private insurance entities.

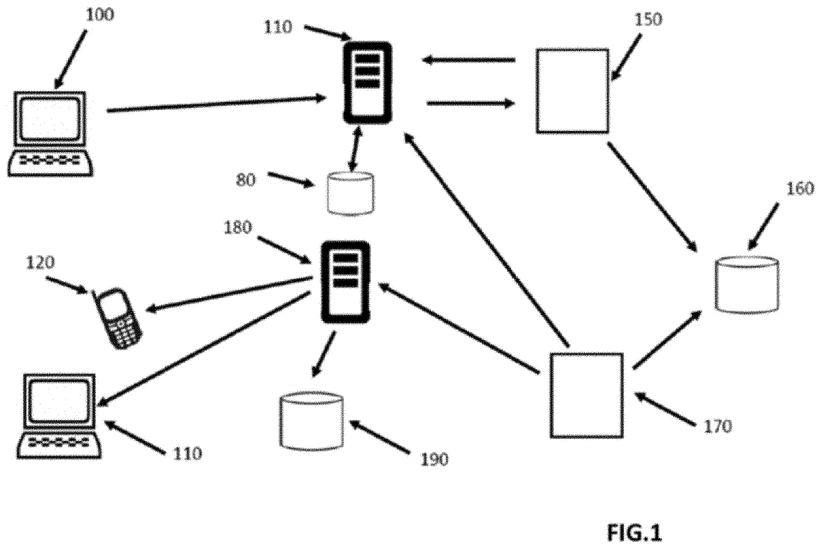

Referring now to the drawings, and more particularly to FIG. 1, there is shown time variable critical path dashboard.

The invention extracts data from the Electronic Medical Record (EMR) database 80 on EMR server 110. The Electronic Medical Record database 80 is the principal data input tool for the system. The invention is capable of working with any Electronic Medical Record system.

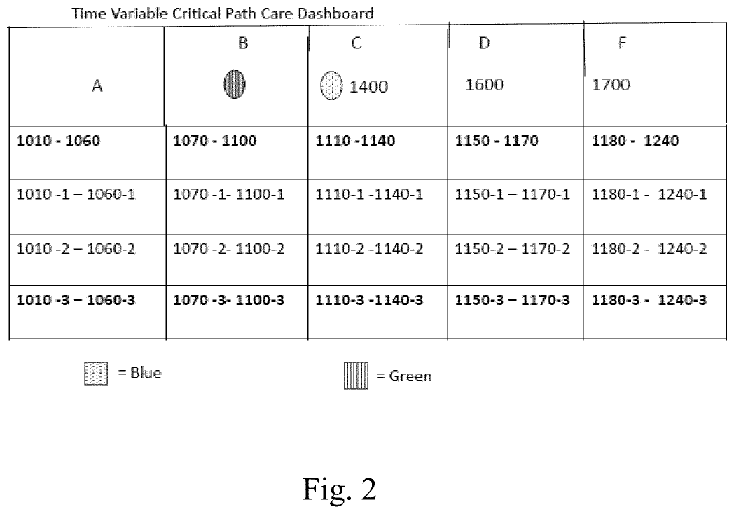

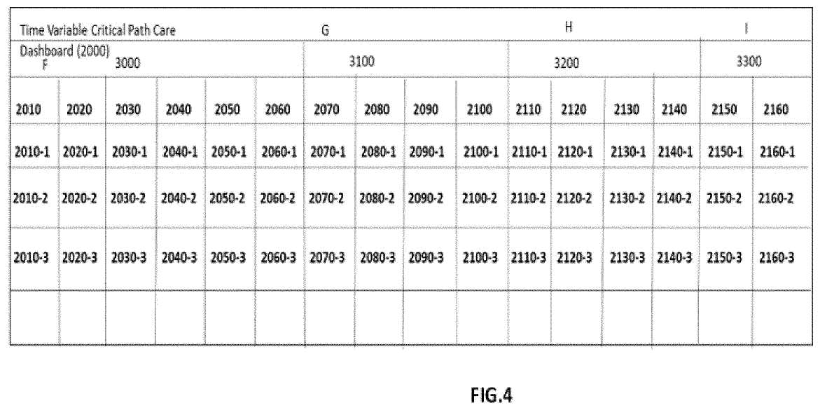

A caregiver places a medical order using work station 100 which sends the medical order using electronic communication to the Electronic Medical Records sever 110 which records the order on Electronic Medical Record database 80. The invention utilizes an interface engine 150 which reads/writes data from the EMR server using an Application Programming Interface (API) on the server and interface engine using electronic communications. The interface engine 150 applies rules stored on database 160 and the data is sent using electronic communications to the Database server 180 and saves the data to storage 190 using electronic communication. The database server 180 can display the information to the dashboard 1000 or 2000 on computer 110 and send SMS text messages to smart phone 120 using electronic communication. Once the data is displayed on the dashboard of the invention 1000 or 2000, the caregiver can read it. Dashboard of the invention 1000 or 2000 is shown in more detail in FIGS. 2 and 4. The electronic communication means can be selected from any one of the following protocols including but not limited to wireless communications such as Wi/FI, Zigbee, RFID, NFC, and Bluetooth as well any form of hardwired communication.

Referring to FIG. 2, there is shown one embodiment of time variable critical path dashboard.

FIG. 2 shows a typical display of time variable critical care dashboard 1000.

Looking at column A. Data field 1010 is the alert field header. Data fields 1010-1, 1010-2 and 1010-3 are where alarms are displayed that correspond to individual patients and their respective rooms. The colored alerts correlate with the color dot next to the diagnosis and in this way, non-clinical caregivers are able to track the patient's critical path. The colors coordinate with the color coding for each diagnosis. For example, chest pain blue, TIA purple. A patient with both diagnoses would have two dots, blue and purple.

Data field 1020 is the room number header. Data fields 1020-1, 1020-2, and 1020-3 have the individual room numbers displayed that correspond to the individual patients.

Data field 1030 is the admission date header. Data fields 1030-1, 1030-2, and 1030-3 have the individual patient's admission date displayed that correspond to the individual patients.

Data field 1040 is the patient name. Data fields 1040-1, 1040-2, and 1040-3 have the individual patients name displayed.

Data field 1050 is the care facility account number. Data fields 1050-1, 1050-2, and 1050-3 have the individual patients account number displayed that correspond to the individual patients.

Data field 1060 is the current time header. Data fields 1060-1, 1060-2, and 1060-3 have the current time displayed. Files 1060-1, 1060-2, and 1060-3 can be combined and only displayed in 1060-1 but this is configurable by caregiving facility.

Column A is the primary key to display the data and it is customizable by the caregiving facility. The facility can track by any of the variables that can be extracted from the primary entry systems such as the Electronic Medical records database. The invention then populates the selected values and enters them into the appropriate cell. The data can be name, account number, or admission date, time zero which is the admission order time by the provider and total length of stay is the time based on the amount of time that has passed from the admission date.

Continuing to column B. In cell 1400 is displayed the symptom that the patient is experiencing such as chest pain, Transient ischemic attack (TIA), Sepsis, Infectious, autoimmune, neurologic, abdominal pain, stroke, myocardial infarction, Syncope, cardiac and respiratory conditions and any other critical care condition. It is customizable by the caregiving facility. The medical order is specific to the symptom and relates to a procedure id which is specific to testing such as Troponin testing, 2-dimensional echo-cardiogram, MRI-B is MRI of the brain, 2-dimensional echo-cardiogram (ultrasound of the heart), Troponin, Carotid ultrasound—ultrasound of the carotid arteries, and electrocardiogram (EKG).

This field is customizable by the caregiving facility. For any of the sub columns 1070, 1080, 1090, and 1100 the invention provides for a pop-up window or an additional cell that shows an alert based on a result not being posted in the allotted time. The invention also displays interval from test order time to test completion, actual test result time, and standard test completion to interpretation time. The invention will also send an alert to the assigned caregiver if the current time from or to completion is greater than 50% of the standard time. The invention also sends an automated message to the consultant when a test is complete.

Data field 1070 is the Troponin testing. Data fields 1070-1, 1070-2, and 1070-3 display the time the Troponin testing was completed for the patient in the specified room. However, in an alternate embodiment it could also display the scheduled time for the procedure.

Data field 1080 is the 2-dimensional echo-cardiogram testing. This is an ultrasound of the heart. Data fields 1080-1, 1080-2, and 1080-3 display the time the 2-dimensional echo-cardiogram testing was completed for the patient in the specified room. However, in an alternate embodiment it could also display the scheduled time for the procedure.

Data field 1090 is the Nuclear stress test field. Data fields 1090-1, 1090-2, and 1090-3 display the time the Nuclear stress testing was completed for the patient in the specified room. However, in an alternate embodiment it could also display the scheduled time for the procedure.

Data field 1100 is the Computed tomography angiography (CTA) test field. Data fields 1100-1, 1100-2, and 1100-3 display the time the CTA testing was completed for the patient in the specified room. However, in an alternate embodiment it could also display the scheduled time for the procedure.

In column B, completion of specific prescribed therapies and procedures are indicated by time in the cell. Non-completion of specific prescribed therapies and procedures are indicated by an empty cell. If the cell has an X in it, it is not applicable for the patient.

Continuing to column C. In cell 1400 is displayed the symptom that the patient is experiencing such as chest pain, Transient ischemic attack (TIA). This field is customizable by the caregiving facility. For any of the sub columns 1110, 1120, 1130, and 1140 the invention provides for a pop-up window or an additional cell that shows an alert based on a result not being posted in the allotted time. The invention also displays the interval from test order time to test completion, actual test result time, and standard test completion to interpretation time. The invention will also send an alert to the assigned caregiver if the current time for or to completion is greater than 50% of the standard time. The invention can also send an automated message to the consultant when a test is complete.

Data field 1110 is the MRI Brain test field. Data fields 1110-1, 1110-2, and 1110-3 display the time the MRI of the brain MRI B testing was completed for the patient in the specified room. However, in an alternate embodiment it could also display the scheduled time for the procedure.

Data field 1120 is the 2-dimensional echo-cardiogram testing. Data fields 1120-1, 1120-2, and 1120-3 display the time of the 2-dimensional echo-cardiogram testing was completed for the patient in the specified room. However, in an alternate embodiment it could also display the scheduled time for the procedure.

Data field 1130 is the Carotid Ultra sound testing. Data fields 1130-1, 1130-2, and 1130-3 display the time the Carotid Ultra sound testing was completed for the patient in the specified room. However, in an alternate embodiment it could also display the scheduled time for the procedure.

Data field 1140 is a customizable testing field which the care facility can use to show information on prescribed therapies and procedures or testing. Data fields 1140-1, 1140-2, and 1140-3 display the time the specific prescribed therapies and procedures testing was completed for the patient in the specified room. However, in an alternate embodiment it could also display the scheduled time for the procedure.

In column C, completion of specific prescribed therapies and procedures are indicated by time in the cell. Non-completion of specific prescribed therapies and procedures are indicated by an empty cell. If the cell has an X in it, it is not applicable for the patient.

Continuing to column D. This column tracks orders placed by consultants and that order is placed in the electronic medical record. This field is customizable by the caregiving facility.

Data field 1150 is customizable testing field which the care facility can use to show information on prescribed therapies and procedures or testing such as Gastroenterology. Data fields 1150-1, 1150-2, and 1150-3 display the time testing was completed for the patient in the specified room for the specific prescribed therapies and procedures. However, in an alternate embodiment it could also display the scheduled time for the procedure.

Data field 1160 is a customizable testing field which the care facility can use to show information on prescribed therapies and procedures or testing such as Cardiology. Data fields 1160-1, 1160-2, and 1160-3 display the time testing was completed for the patient in the specified room for the specific prescribed therapies and procedures. However, in an alternate embodiment it could also display the scheduled time for the procedure.

Data field 1170 is a customizable testing field which the care facility can use to show information on prescribed therapies and procedures or testing such as Neurology. Data fields 1170-1, 1170-2, and 1170-3 display the time testing was completed for the patient in the specified room for the specific prescribed therapies and procedures. However, in an alternate embodiment it could also display the scheduled time for the procedure.

Continuing to column E. This column tracks orders' time and discharge information of orders placed in the electronic medical record. This field is customizable by the caregiving facility.

Data field 1180 displays the time since admission in days, hours, and seconds. This is the difference between the current date and time and the admission date and time. Data fields 1180-1, 1180-2, and 1180-3 display the length of the time since admission for each patient.

Data field 1190 displays the Length of stay and has three different states. Green is within payer contracted time, yellow is the time is close to reaching the maximum, and red is that the patient has stayed longer than the maximum. This is the difference between the current date and time and the admission date and time and it looks at the payer reimbursement amount to determine the maximum length of stay that is covered under the insurance plan. Data fields 1180-1, 1180-2, and 1180-3 are the color code for each patient.

Data field 1200 displays the scheduled time of the discharge conditional order time. This the current date and time when discharge is scheduled. Data fields 1200-1, 1200-2, and 1200-3 display the time when discharge is scheduled for each patient.

Data field 1210 displays the final ordered scheduled time of the discharge final order time. This is the current date and time when discharge final order is scheduled. Data fields 1210-1, 1210-2, and 1210-3 display the time when discharge final order is scheduled for each patient.

Data field 1220 displays the actual time of the discharge for the patient. This the current date and time when discharge was final. Data fields 1220-1, 1220-2, and 1220-3 display the time when discharge was final for each patient.

Looking at some specific entries:

If a cell is empty that means that the patient diagnosis is pending, if the patient has chest pain, the indicator is red. The indicator is blue if TIA and an X means no value is expected.

The invention looks at the patient stay as a time gradient event which ages as the time of stay gets longer. This is to alert the caregivers that the patient is either within the targeted stay for or is approaching or exceeding the allotted time. This is an important tool for alerting different caregivers on different shifts about the issue associated with time that is not reimbursable. An example: The patient provider has a goal of determining a diagnosis in 20 hours. The display would be shown as follows: if the time of stay is less than 5 hours the alert is shown in cell 1190 length of stay indicator would be green, if the stay was greater than 5 hours but less than 10 hours, length of stay indicator would be green/yellow, if the stay was greater than 10 hours but less than 15 hours, length of stay indicator would be yellow, if the stay was greater than 15 hours but less than 18 hours, length of stay indicator would be amber and if the stay was greater than 18 hours, the indicator would be red. The indicator alerts are all customizable by the care facility.

The time variable critical path shows the caregiver by alerts and times displayed, which specific prescribed therapies and procedures for the patient in the specified room, what is not required, and what is needed to be completed. If a procedure is not completed in a timely fashion, then this could result in delaying moving the patient to the next phase of their treatment.

The invention allows for customizable triggers for all events, so each care facility can customize them for their own policies and procedures.

The invention allows multiple ways to communicate issues and alarms based on the customizable triggers. It can send alerts to the caregivers by text message, email, EMR messaging system, or any other source like cellular phones.

The invention is applicable for use by all disciplines within the caregiver facility.

The invention can be used as a source of information for analytics or other business intelligence programs as well as data for new evidence-based protocols to improve patient care and speed up the care process.

The Critical path are the elements that are evidence-based and must be completed to determine the total length of stay. The invention can provide alarms to caregivers to review patient specific prescribed therapies and procedures to recommend process changes and promotes the ability for care givers to implement customizable triggers for individual patients as the need requires.

The invention can also support advance analytics for the caregiver facility:
a. The invention can support customizable analysis dynamics;
b. The invention allows caregivers to drill down to time intervals to determine which elements contribute most to total Length of Stay (LOS);
c. The invention allows caregivers to look at critical path elements more expeditiously by reviewing the comments to analyze in terms of frequency time frames to facilitate process differences;
d. The invention allows caregivers the ability to place comments onto the dashboard as a source of data for the process change. An example is a Length of Stay (LOS) greater than X hours despite completion of critical path;
e. The invention provides an avenue for employee feedback that can be built into analytics for caregiver facility management use;
f. The invention allows for customized metrics for management use such as MRI order time, to MRI read time with time frames, intervals, evaluate contribution to total LOS, and quantify in terms of dollars to justify more resources;
g. The invention allows caregivers to predict analytics and what if scenarios. An example is what if we decrease time to complete a test by a certain time interval, what would happen to the total LOS.

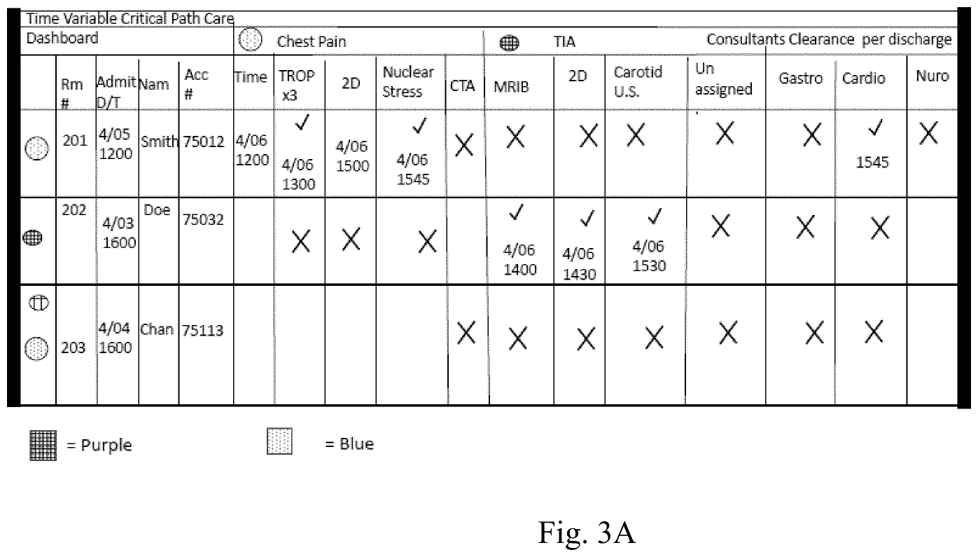

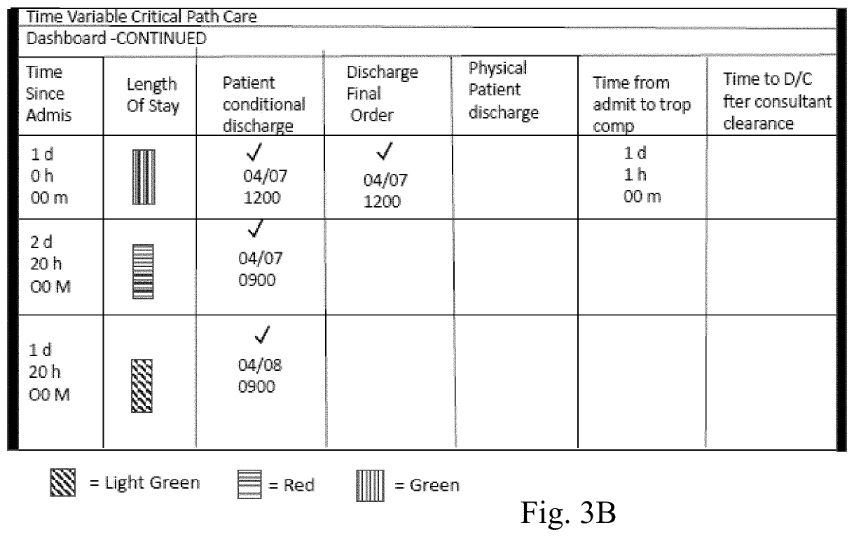

Referring to FIG. 3 which shows a populated dashboard 1000 of the invention is shown. The dashboard 1000 can alternatively be called a dashboard display and within the disclosure, the terms are used interchangeably. In the alternative dashboard/dashboard display, the colored dots correlate with the color dot next to the diagnosis—in this way, non-clinical caregivers are able to track the patient's critical path. The colors coordinate with the color coding for each diagnosis for example chest pain blue, TIA-purple. A patient with both diagnoses would have two dots, blue and purple.

FIG. 3 shows a typical dashboard of the invention as it would be displayed to the caregivers. Specifically looking at the patient in room 201 we can readily see that the patient was admitted on April 5 at 1200 and the current date and time is Apr. 6, 2019 at 1200 and that the patient has been in the care facility for 1 day and has had the following procedures completed for chest pain diagnosis, Troponin testing was completed at 1300 today, 2 dimensional echo-cardiogram testing was completed at 1500 today, Nuclear stress test was completed at 1545 today and a Cardiovascular specialist has cleared the patient at 1545 today. We also know that the patient length of stay is within the normal range and there is a conditional discharge order scheduled for Apr. 7, 2019 at 1200 and a final discharge order scheduled for April 7 at 1200.

Alternatively, looking at the patient in room 202 we can see that the admission date was Apr. 3, 2019 at 1600 and has had the following testing completed. The patient had a Computed tomography angiography (CTA) test completed at 1400 today for chest pain diagnosis and 2-dimensional echo-cardiogram testing completed at 1430 and a Carotid Ultra Sound test completed at 1530 for Transient ischemic attack (TIA) diagnoses. We also note that Non-completion indicated by an empty cell of a prescribed MRI and a number of therapies and procedures are indicated as not applicable for the patient, being indicated by an X in the cell. The check above the time means that the procedure is complete. In some embodiments the check symbol can be displayed in green to convey that the procedure was done on schedule or red to convey that the procedure was delayed and not completed on schedule. The caregiver can identify which test or procedures are missing, late or not prescribed. This allows the caregiver to manage the caregiving process to expedite the care and maintain the patient on the pre-agreed to process with the payers, care facility management, and best practices.

Referring to FIG. 4, there is shown an alternative embodiment of Time variable critical path dashboard 2000.

Looking at column F. Data field 3000 is the heading field and is optional. Data field 2010 is the alert field header. Data fields 2010-1, 2010-2, and 2010-3 are where alarms are displayed that correspond to individual patients and their respective rooms.

Data field 2020 is the room number header. Data fields 2020-1, 2020-2, and 2020-3 have the individual room numbers displayed that correspond to the individual patients.

Data field 2030 is the Length of Stay header. Data fields 2030-1, 2030-2, and 2030-3 have the individual patients Length of Stay displayed that correspond to the individual patients. The Length of stay can be displayed in hours, hours and minutes, or days, hours and minutes and is configurable by the care facility.

Data field 2040 is the admission date header. Data fields 2030-1, 2030-2, and 2030-3 have the individual patients' admission date displayed in month, day, year, hours, and minutes that correspond to the individual patients.

Data field 2050 is the systolic blood pressure indicator. Data fields 2050-1, 2050-2, and 2050-3 have the time and date displayed if a patient's systolic blood pressure is lower than a customizable set point. Each patient set point is customizable or the care facility can set a facility specified minimum systolic blood pressure. A preferred systolic blood pressure lower limit for the invention is 85 however the set point is customizable and can be set to any evidence-based value.

Data field 2060 is the Heart Rate indicator. Data fields 2060-1, 2060-2, and 2060-3 have the time and date displayed if a patient's heart rate is greater than a customizable set point. Each patient set point is customizable or the care facility can set a facility specified maximum heart rate. A preferred heart rate upper limit for the invention is 130 however the set point is customizable and can be set to any evidence-based value.

Column F is the primary key to display the data and it is customizable by the caregiving facility. The facility can track by any of the variables that can be extracted from the primary entry systems such as the Electronic Medical records database. The invention then populates the selected values and enters them into the appropriate cell. The data can be systolic blood pressure limit time, heart rate upper limit time, name, account number or admission date, Time zero which is the admission order time by the provider, and Total length of stay is the time based on the amount of time that has passed from the admission date.

Turning our attention to Column G, Data field 3100 is the primary key to display the data and it is customizable by the caregiving facility. The facility can track by any of the variables that can be extracted from the primary entry systems such as the Electronic Medical records database. The invention then populates the selected values and enters them into the appropriate cell. The data can be a 2-dimensional echo-cardiogram test time, electrocardiogram test, Troponin test time, stress test time, or any relevant test.

Continuing in column G. In cell 3100 is displayed the symptom that the patient is experiencing such as chest pain, Transient ischemic attack (TIA), Sepsis, Infectious, autoimmune, neurologic, cardiac, and respiratory conditions and any other critical care condition. It is customizable by the caregiving facility. This field is customizable by the caregiving facility. For any of the sub columns 2070, 2080, 2090, and 2100 the invention provides for a pop-up window or an additional cell that shows an alert based on a result not being posted in the allotted time. The invention also displays the interval from test order time to test completion, actual test result time and standard test completion to interpretation time. The invention will also send an alert to the assigned caregiver if the current time from or to completion is greater than 50% of the standard time. The invention can also send an automated message to the consultant when a test is complete.

The medical order is specific to the symptom and relates to a procedure id which is specific to testing such as Troponin testing, 2-dimensional echo-cardiogram, MRI-B is MRI of the brain, 2-dimensional echo-cardiogram (ultrasound of the heart), Troponin, Carotid ultrasound—ultrasound of the carotid arteries and electrocardiogram (EKG).

Data field 2070 is the electrocardiogram (EKG). Data fields 2070-1, 2070-2, and 2070-3 display the time the EKG testing was completed for the patient in the specified room for the specific procedure. However, in an alternate embodiment it could also display the scheduled time for the procedure.

Data field 2080 is the Troponin testing. Data fields 2080-1, 2080-2, and 2080-3 display the time the Troponin testing was completed for the patient in the specified room for the specific procedure. However, in an alternate embodiment it could also display the scheduled time for the procedure.

Data field 2090 is the 2-dimensional echo-cardiogram testing. This is an ultrasound of the heart. Data fields 2090-1, 2090-2, and 2090-3 display the time the 2-dimensional echo-cardiogram testing was completed for the patient in the specified room for the specific procedure. However, in an alternate embodiment it could also display the scheduled time for the procedure.

Data field 2100 is the Stress test field. Data fields 2100-1, 2100-2, and 2100-3 display the time the Stress testing was completed for the patient in the specified room for the specific procedure. However, in an alternate embodiment it could also display the scheduled time for the procedure.

In column G, completion of specific prescribed therapies and procedures are indicated by time in the cell. Non-completion of specific prescribed therapies and procedures are indicated by an empty cell. If the cell has an X in it, it is not applicable for the patient.

Continuing to column H. In cell 3200 is information about clearance specific to post discharge events. This field is customizable by the caregiving facility. For any of the sub columns 2110, 2120, 2130, 2140, 2150, and 2160 the invention provides for a pop-up window or an additional cell that shows and alert based on a result not being posted in the allotted time. The invention also displays the interval from test order time to test completion, actual test result time, and standard test completion compared to actual interpretation time. The invention will also send an alert to the assign caregiver if the current time for or to completion is greater than 50% of the standard time. The invention also sends an automated message to the consultant when a test is complete.

Data field 2110 is the Transport Requested field. Data fields 2110-1, 2110-2, and 2110-3 display the time the Transport is requested for the patient in the specified room. However, in an alternate embodiment it could also display the scheduled time for the transport.

Data field 2120 is the Cardio clearance field. Data fields 2120-1, 2120-2, and 2120-3 display the time the cardiologist has cleared the patient in the specified room. However, in an alternate embodiment it could also display the scheduled time for the cardiologist to see the patient.

Data field 2130 is the Primary Care Physician field. Data fields 2130-1, 2130-2, and 2130-3 display the time when the appointment was made for the patient to see the Primary Care Physician for the patient in the specified room.

Data field 2140 is Computerized physician order entry (CPOE) which is a process of electronic entry of medical practitioner instructions for the treatment of patients. This is a customizable field which the care facility can use to show information on prescribed therapies and procedures or testing. Data fields 2140-1, 2140-2, and 2140-3 display the time CPOE orders were placed for the patient in the specified room.

In column I, cell 3300 the current time is displayed.

Data field 2150 is the Length of Stay header. Data fields 2150-1, 2150-2, and 2150-3 have the individual patients Length of Stay displayed that correspond to the individual patients. The Length of stay can be displayed in hours, hours and minutes, or days, hours, and minutes and is configurable by the care facility.

Data field 2160 is the room number header. Data fields 2160-1, 2160-2, and 2160-3 have the individual room numbers displayed that correspond to the individual patients.

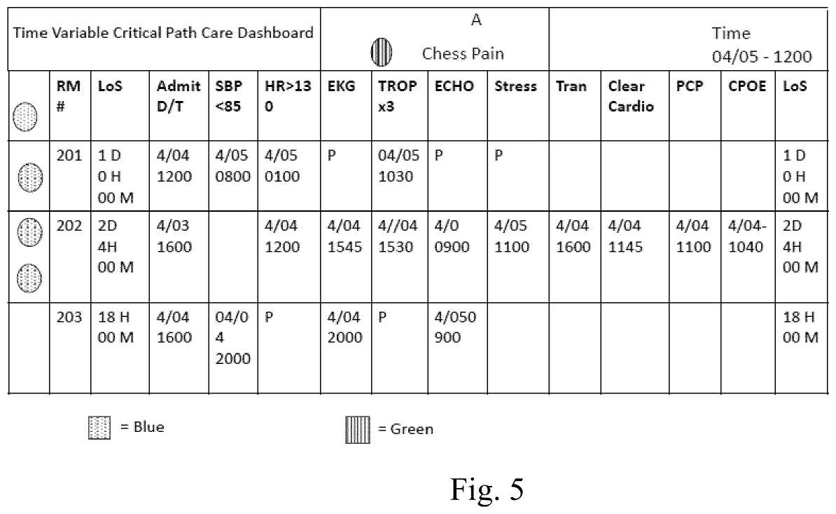

Referring to FIG. 5 which shows a populated dashboard 2000 of the alternative embodiment of the invention. The dashboard 2000 can alternatively be called a dashboard display and within the disclosure the terms are used interchangeably. The alternative dashboard/dashboard display the colored dots correlate with the color dot next to the diagnosis—in this way, non-clinical caregivers are able to track the patient's critical path. The colors coordinate with the color coding for each diagnosis for example chest pain blue, TIA-purple. A patient with both diagnoses would have two dots, blue and purple.

FIG. 5 shows a typical dashboard alternative embodiment of the invention as it would be displayed to the caregivers. Specifically looking at the patient in room 201 we can readily see that the patient was admitted on April 4 at 1200 and the current date and time is Apr. 5, 2019 at 1200 and that the patient has been in the care facility for 1 day and has had the following procedures completed for chest pain diagnosis, Troponin testing was completed at 1030 today, 2 dimensional echo-cardiogram testing is pending, stress test is pending, and an electrocardiogram (EKG) is pending. The patient had a low systolic blood pressure on April 05 at 0800 and a high blood pressure reading on April 05 at 0100. We also know that the patient length of stay is within the normal range.

Alternatively, looking at the patient in room 202 we can see that the admission date was Apr. 3, 2019 at 1600 and has had the following testing completed for chest pain diagnosis. The patient has had, Troponin testing that was completed on April 4 at 1530 t, 2-dimensional echo-cardiogram testing on April 5 at 0900, stress test on April 5 at 1100, and an electrocardiogram (EKG) on April 04 at 1545. The patient had a low systolic blood pressure on April 5 at 0800 and a high blood pressure reading on April 5 at 0100. The patient also had a high blood pressure reading on April 4 at 1500. We also know that the patient length of stay is within the normal range. The caregiver can identify which test or procedures are missing, late, or not prescribed. This allows the caregiver to manage the caregiving process to expedite the care and maintain the patient on the pre-agreed to process with the payers, care facility management, and best practices.

We also note that Non-completion indicated by an empty cell of a prescribed MRI and a number of therapies and procedures are indicated as not applicable for the patient being indicated by an X in the cell. The check above the time means that the procedure is complete. In some embodiments the check symbol can be displayed in green to convey that the procedure was done on schedule or red to convey that procedure was delayed and not completed on schedule.

As one can see from FIG. 5 the caregiver can look at the dashboard display. The invention which is manifested by the dashboard/dashboard display allows the caregiver to see which procedures associated with each procedure id have been completed, which are pending, and which are delayed. This allows the caregiver to take the appropriate action to ensure that the patient is released within the prescribed times set by the payers, hospital administrators, best clinical practices, and caregiver knowledge.

By ensuring that all diagnostic procedures have been completed within a fixed period, it ensures that the patient receives the various tests, procedures, and therapies prescribed by the caregiver, hospitalist, and consultants such as the neurologist and cardiologist, that need to provide an informed diagnosis and it ensures that the care facility does not have to keep the patient for an extended length of stay where the extended time period is not reimbursed by the payer which include state, federal, and private insurance entities.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method for a caregiver to monitor medical procedures with respect to at least one patient in a hospital comprising:
said caregiver having a smartphone wherein said smartphone is enabled to receive SMS text messages;
said caregiver having visual access to a dashboard display;
said caregiver entering at least one medical order specific to one symptom on a remote computer electronically in communication with an electronic medical record server;
said electronic medical record server storing said at least one medical order in an electronic medical record database on a storage media electronically in communication with said electronic medical record server;
said electronic medical record database is in electronic communication with an interface engine on said remote computer using said electronic medical record server;
said interface engine on said remote computer is in electronic communication with a rules database on said remote computer and said rules database storing rules for using said at least one medical order stored in said electronic medical record database;
said interface engine on said remote computer applying said rules to said at least one medical order and said rules creating at least one dashboard data set comprising of an alert, time and procedure id;
said interface engine being electronically in communication with a second database server and said second database server is in electronic communication with dashboard data to storage;
said second database server storing said at least one dashboard data set on said dashboard data to storage on said remote computer;
said second database server is in electronic communication with said dashboard display and said second database server communicating said at least one dashboard data set comprising of said alert, said time and said procedure id to said dashboard display;
said second database server being in electronic communication with said smart phone or tablet and communicating said at least one dashboard data set using said SMS text messages and said SMS text messages comprising said alert, said time and said procedure id to said smartphone or tablet; said caregiver reading said at least one dashboard data set on said smart phone or tablet and said dashboard display; and
said caregiver identifying said procedure id and confirming completion time of said procedure id and said caregiver entering orders for said at least one patient discharge from said hospital.

2. The electronic communication of claim 1, selected from the group consisting of Wi/FI, Zigbee and Bluetooth.

3. The symptom of claim 1, selected from the group consisting of chest pain, Transient ischemic attack (TIA), Sepsis, abdominal pain, Stroke, myocardial infarction, Infectious, autoimmune, neurologic, cardiac and respiratory conditions.

4. The procedure id of claim 1, selected from the group consisting of Troponin testing, 2-dimensional echo-cardiogram, MRI-B is MRI of the brain, 2-dimensional echo-cardiogram (ultrasound of the heart), Troponin, Carotid ultrasound—ultrasound of the carotid arteries and electrocardiogram (EKG).

5. The dashboard display of claim 1, wherein dashboard display is capable of displaying more than one dashboard data set and said dashboard being readable by said caregiver.

6. The dashboard display capable of displaying more than one dashboard data set of claim 5, wherein the caregiver reads more than one dashboard data set to identify missing dashboard data sets.

7. The dashboard display capable of displaying more than one dashboard data set of claim 5, wherein the caregiver reads more than one dashboard data set to identify dashboard data sets wherein said time is late.

8. A method for monitoring medical procedures comprising:
entering at least one medical order specific to one symptom on a remote computer electronically in communication with an electronic medical record server;
said electronic medical record server storing said at least one medical order in an electronic medical record database on a storage media electronically in communication with said electronic medical record server;
the electronic medical record database is in electronic communication with an interface engine using said electronic medical record server;
said interface engine is in electronic communication with a rules database and said rules database storing rules for using said at least one medical order stored in said electronic medical record database;
said interface engine applying said rules to said at least one medical order said rule creating at least one dashboard data set comprising of an alert, time and procedure id;
said interface engine being electronically in communication with a second database server and said second database server is in electronic communication with dashboard data to storage;
said second database server is in electronic communication with a dashboard display and said second database server communicating to said dashboard display said at least one dashboard data set;
said second database server being in electronic communication with a smart phone or tablet and communicating said at least one dashboard data set to said smartphone or tablet;
reading said at least one dashboard data set on said smart phone or tablet and said dashboard display; and
identifying said procedure id that has been completed.

9. The electronic communication of claim 8, selected from the group consisting of Wi/FI, Zigbee and Bluetooth.

10. The symptom of claim 8, selected from the group consisting of chest pain, Transient ischemic attack (TIA), Sepsis, abdominal pain, Stroke, myocardial infarction, Infectious, autoimmune, neurologic, cardiac and respiratory conditions.

11. The procedure id of claim 8, selected from the group consisting of Troponin testing, 2-dimensional echo-cardiogram, MRI-B is MRI of the brain, 2-dimensional echo-cardiogram (ultrasound of the heart), Troponin, Carotid ultrasound—ultrasound of the carotid arteries and electrocardiogram (EKG).

12. The dashboard display of claim 8, wherein said dashboard display is capable of displaying more than one dashboard data set and said dashboard being readable by a caregiver.

13. The dashboard display capable of displaying more than one dashboard data set of claim 12, wherein a caregiver reads more than one dashboard data set to identify missing dashboard data sets.

14. The dashboard display capable of displaying more than one dashboard data set of claim 12, wherein a caregiver reads more than one dashboard data set to identify dashboard data sets wherein said time is late.

15. The dashboard display of claim 8, wherein the at least one dashboard data set procedure id is associated with an alert, time and procedure id.

16. The time of claim 15, wherein said time represents the time of completion of the procedure associated with said procedure id.

17. A system for monitoring medical procedures comprising:
an electronic medical record server storing at least one medical order and one blood pressure reading of at least one patient in an electronic medical record database on a storage media electronically in communication with said electronic medical record server;
the electronic medical record database is in electronic communication with an interface engine using said electronic medical record server;
said interface engine is in electronic communication with a rules database and said rules database storing rules for using said at least one medical order stored in said electronic medical record database;
said interface engine applying said rules to said one blood pressure reading of at least one patient and setting a blood pressure alarm based on said rules;
said interface engine applying said rules to said at least one medical order and said rules creating at least one dashboard data set comprising of a blood pressure alarm indicator, alert, time and procedure id;
said interface engine being electronically in communication with a second database server and said second database server is in electronic communication with dashboard data to storage;
said second database server storing said at least one dashboard data set on said dashboard data to storage;
said second database server is in electronic communication with a dashboard display and said second database server communicating said at least one dashboard data set comprising of said alert, said time and said procedure id to said dashboard display;
said second database server being in electronic communication with a smart phone or tablet and communicating said at least one dashboard data set comprising of said alert, said time and said procedure id to said smartphone or tablet; and
wherein a caregiver can enter at least one medical order specific to one symptom on the remote computer electronically in communication with the electronic medical record server.

18. The electronic communication of claim 17, selected from the group consisting of Wi/FI, Zigbee and Bluetooth.

19. The symptom of claim 17, selected from the group consisting of chest pain, Transient ischemic attack (TIA), Sepsis, abdominal pain, Stroke, myocardial infarction, Infectious, autoimmune, neurologic, cardiac and respiratory conditions.

20. The procedure id of claim 17, selected from the group consisting of Troponin testing, 2-dimensional echo-cardiogram, MRI-B is MRI of the brain, 2-dimensional echo-cardiogram (ultrasound of the heart), Troponin, Carotid ultrasound—ultrasound of the carotid arteries and electrocardiogram (EKG).
